(19) United States
(12) Patent Application Publication
(10) Pub. No.: US 2012/0176241 A1
(43) Pub. Date: Jul. 12, 2012

(54) Door Handle Sterilizer

(75) Inventors: Scott Pasch, Warren, NJ (US); Edward J. Laganis, Hoboken, NJ (US)

(73) Assignee: Scott Pasch, Warren, NJ (US)

(21) Appl. No.: 13/345,980

(22) Filed: Jan. 9, 2012

Related U.S. Application Data
(60) Provisional application No. 61/431,201, filed on Jan. 10, 2011.

(51) Int. Cl. G08B 21/00; A61L 2/10; G01J 1/44; E05B 1/00

(52) U.S. Cl. 340/540; 16/412; 250/492.1; 250/214.1

(57) ABSTRACT

A method for sterilizing a door handle is provided. A shield and a door handle are positioned into a first relative position whereby the shield protects a user from a germicide process. A germicide process is activated. The shield and the door handle are positioned into a second relative position at the conclusion of the germicide process whereby the door handle is accessible to the user to open the door.

The present application claims the benefit of U.S. Provisional Patent Application Ser. No. 61/431,201, filed Jan. 10, 2011, which is incorporated herein by reference.

### TECHNICAL FIELD

This invention relates generally to sterilization of door handles and more particularly to sterilization of a door handle through exposure to ultraviolet light.

### BACKGROUND

Many of the germs associated with disease are transmitted through touching and especially touching hands. Among the most frequently touched items in a day, door handles of doors in public spaces are contacted by a great many people. As each person grabs a door handle germs from that person's hands remain on the surface and are available to be passed to the next person who grabs the door handle and possibly even other people later in the day. Secondary contact is the transmission of germs from one hand to the next via touching a common object such as a door handle.

Some people have begun to take preventive steps against secondary contact, such as washing their hands or using a disinfecting liquid such as Purell™ to mitigate against germ propagation. These preventative measures are impractical for most people to use each time a commonly handled surface is touched. Bathrooms are areas where there is significant danger of germs transmission. Most people wash their hands after using bathroom facilities, but use their clean hands to touch the doorknob upon leaving the bathroom. A bathroom doorknob may contain bacteria or other germs left by a prior bathroom user who did not adequately wash their hands. A person who just used the bathroom door knob will often not have another opportunity to wash their hands until they return to the bathroom. Moreover, other doorknobs in public spaces can often be touched by various people who may provide exposure to contagious diseases. It is not practical for every person using the public space to wash their hands or use a sanitizer after every contact with a doorknob.

### SUMMARY

In accordance with an embodiment, a method for sterilizing a door handle is provided. A shield and a door handle are positioned into a first relative position whereby the shield protects a user from a germicide process. A germicide process is activated. The shield and the door handle are positioned into a second relative position at the conclusion of the germicide process whereby the door handle is accessible to the user to open the door.

In accordance with an embodiment, a door handle system is provided. The door handle system comprises a door handle, a shield, and a germicide source configured to disinfect the door handle. The door handle system further comprises a driver configured to change the relative position of the door handle and the shield such that a user is shielded during application of the germicide source and such that the door handle is accessible to the user when the germicide source is not applied.

In accordance with an embodiment, an apparatus that is configured to attach to a door comprising a door handle is provided. The apparatus comprises a bracket configured to attach to the door, a shield, and a germicide source configured to disinfect the door handle. The apparatus further comprises a driver configured to change the relative position of the door handle and the shield such that a user is shielded during application of the germicide source and such that the door handle is accessible to the user when the germicide source is not applied.

These and other advantages of the present disclosure will be apparent to those of ordinary skill in the art by reference to the following Detailed Description and the accompanying drawings.

### BRIEF DESCRIPTION OF THE DRAWINGS

FIG. 1 shows a door configured with a door handle sterilizer in accordance with an embodiment;

FIG. 2 shows functional components of an exemplary controller in accordance with an embodiment;

FIG. 3 shows a flowchart for a method of interacting with a door in accordance with an embodiment;

FIG. 4 shows a flowchart for a method of interacting with a door in accordance with an embodiment;

FIG. 5 shows a system comprising a door configured with a door handle sterilizer apparatus mounted via a bracket in accordance with an embodiment;

FIG. 6 shows a door handle sterilizer apparatus in accordance with an embodiment;

FIG. 7a-c show a door configured with a door handle sterilizer in accordance with an embodiment;

FIG. 8a-c show a user interacting with a door configured with a door handle sterilizer in accordance with an embodiment; and

FIG. 9 shows components of an exemplary computer that may be used to implement the system described herein.

### DETAILED DESCRIPTION

In accordance with an embodiment, a method for sterilizing a door handle is provided. A shield and a door handle are positioned into a first relative position whereby the shield protects a user from a germicide process. A germicide process is activated. The shield and the door handle are positioned into a second relative position at the conclusion of the germicide process whereby the door handle is accessible to the user to open the door.

In accordance with an embodiment, a door handle system is provided. The door handle system comprises a door handle, a shield, and a germicide source configured to disinfect the door handle. The door handle system further comprises a driver configured to change the relative position of the door handle and the shield such that a user is shielded during application of the germicide source and such that the door handle is accessible to the user when the germicide source is not applied.

In accordance with an embodiment, an apparatus that is configured to attach to a door comprising a door handle is provided. The apparatus comprises a bracket configured to attach to the door, a shield, and a germicide source configured to disinfect the door handle. The apparatus further comprises a driver configured to change the relative position of the door handle and the shield such that a user is shielded during application of the germicide source and such that the door handle is accessible to the user when the germicide source is not applied.

In accordance with certain embodiments, the germicide process comprises illuminating an ultraviolet light. In accordance with certain embodiments, positioning a shield and a handle into a first relative position comprises moving the shield while the door handle remains stationary. In accordance with certain embodiments the user of the door handle may be alerted that the door handle is accessible to the user to open the door.

In accordance with an embodiment, a sensor may sense an object in proximity to the door and generate a proximity signal based on the sensing. In accordance with an embodiment, a safety override signal may be activated based on the proximity signal. In accordance with certain embodiments, information associated with the germicide process may be received and a status message may be transmitted via a network, the status message comprising information associated with the germicide process.

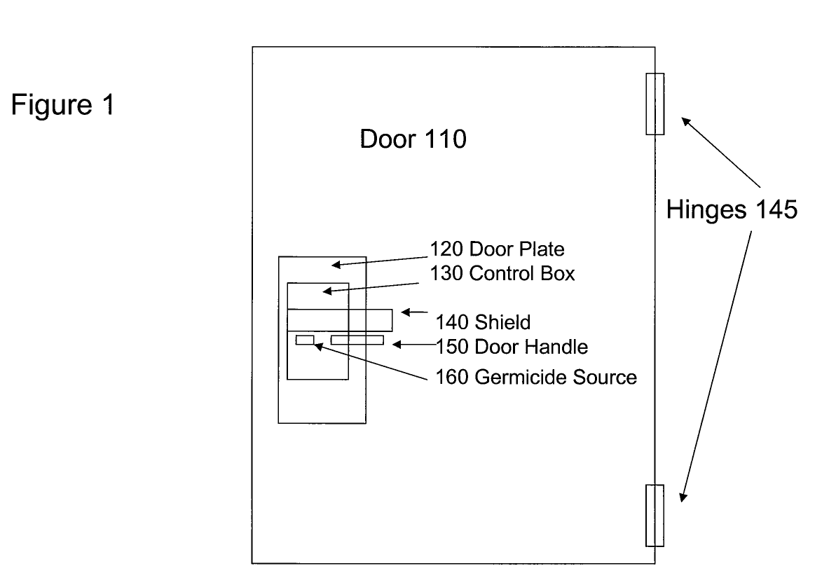

In an exemplary embodiment, illustrated in FIG. 1, door 110 is mounted in a doorway on hinges 145. Door plate 120 is attached to door 110. Door plate 120 is further attached to control box 130, shield 140, door handle 150, and germicide source 160. In an exemplary embodiment, control box 130 is mechanically connected to one or both of shield 140 and door handle 150. Control box 130 may be configured to position one or both of shield 140 and door handle 150 in a plurality of relative positions.

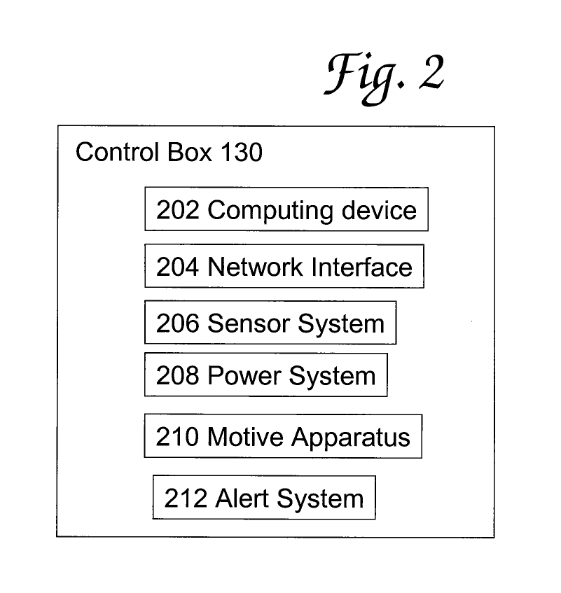

In an exemplary embodiment, illustrated in FIG. 2, control box 130 may comprise components such as: computing device 202, network interface 204, sensor system 206, power system 208, motive apparatus 210, and alert system 212. Computing device 202 may be operable to store computer program instructions which, when executed on a processor, cause the processor to control the functioning of the various components in accordance with an embodiment of the invention. Network interface 204 may be operable to communicate with external devices regarding the status of components such as control box 130, shield 140, door handle 150, and germicide source 160. In certain embodiments, network interface 204 may communicate via a wireless network, a wired network or LAN, a Bluetooth network or other communications networks appropriate for communication between computing devices.

In certain embodiments sensor system 206 may comprise a plurality of sensors. The plurality of sensors may comprise sensors of various types, for example: a sensor to detect the state of the apparatus, a sensor to detect the state of the germicide process, a proximity sensor to detect an object in proximity to door handle 150. In an embodiment where germicidal source 160 comprises a consumable material (such as a disinfectant liquid or a light source with a finite lifetime) sensor system 206 may comprise a sensor to detect a level of the consumable material. Based on the level of the consumable material, control box 130 may generate a status message comprising information associated with the germicide process. In certain embodiments, sensor system 206 may comprise a sensor that detects information regarding the germicide process.

In an exemplary embodiment, control box 130 may comprise power system 208. In certain embodiments power system 208 may receive power via a connection to the electrical system of a building in which it operates. In certain embodiments, power system 208 may receive power from a wireless source. In certain embodiments, power system 208 may receive power from a solar cell or a similar local power source.

In an exemplary example, control box 130 may comprise motive apparatus 210 configured to position shield 140 and door handle 150 so that they may be moved from a first relative position to a second relative position. In certain embodiments, shield 140 may remain stationary and door handle 150 may move from a first position relative to shield 140 to a second position relative to shield 140 in response to an impulse received from motive apparatus 210. In certain embodiments, door handle 150 may remain stationary and shield 140 may move from a first position relative to door handle 150 to a second position relative to door handle 150 in response to an impulse received from motive apparatus 210. In certain embodiments, both of door handle 150 may and shield 140 may each move into positions such that door handle 150 and shield 140 start from a first position relative position with respect to each other and are re-positioned into to a second position relative to each other.

In certain embodiments, control box 130 may comprise alert system 212. Alert system 212 may be configured to indicate to a user interacting with door handle 150 that germicide source 160 is operating. In an embodiment, alert system 212 may activate to alert a user at the commencement of the germicide process and terminate at the end of the germicide process. In certain embodiments alert system 212 may provide an indication if the sterilization system is malfunctioning or if door handle 150 is not sterilized.

In certain embodiments, germicide source 160 may emit an active ingredient that is toxic to germs. In an exemplary embodiment germicide source 160 may emit ultraviolet light as an active ingredient. In certain embodiments, germicide source 160 may emit a disinfectant substance in liquid or gaseous form that is operative to kill germs or otherwise prevent the spread of germs. In an exemplary embodiment, germicide source 160 may comprise an ultraviolet light that provides ultraviolet light whose frequency has been selected to efficiently disinfect the surface of door handle 150. An example of an ultraviolet light wavelength range that could be used in germicide source 160 would be 280 nm-100 nm. Ultraviolet light with a wavelength of 280 nm-100 nm may sometimes be referred to as ultraviolet-C light. Light of different wavelengths may be used depending on the specific design parameters of a particular embodiment of a germicide source. For many germicidal applications, ultraviolet light of wavelength 254 nm is used. Generally a design choice of a light source that supplies light with a wavelength of 254 nm is appropriate for use in an exemplary germicide source.

In an exemplary embodiment, germicide source may be activated by an illuminate signal that is received from control box 130 and may be deactivated by a termination signal that is received from control box 130. In certain embodiments germicide source 160 may comprise a combination of disinfecting agents. For example, in certain embodiments, germicidal source may provide heat to the surface of door handle 150. In certain embodiments, germicidal source 160 may spray a disinfectant liquid onto door handle 150. In certain embodiments germicidal source 160 may utilize several disinfectant agents in combination, where the combination may include two or more agents such as heat, a liquid, a gas, ultraviolet light, or another suitable agent.

In certain embodiments, germicidal source 160 may execute a germicide process to disinfect door handle 150. For example, in an embodiment where germicidal source 160 comprises an ultraviolet light, a germicide process may be a time period during which door handle 150 is illuminated with the ultraviolet light. In an embodiment where germicidal source 160 comprises a source of another consumable such as a material disinfectant, a germicide process may comprise an amount of material applied to door handle 150.

In certain embodiments, shield 140 may be selected to have characteristics appropriate for a particular germicide source 160. For example, in an embodiment in which into germicide source 160 comprises an ultraviolet light source, shield 140 may be a material that will block ultraviolet light. Generally, shield 140 may be selected to prevent leakage of the active ingredient from germicide source 160. Thus, shield 140 may prevent the active ingredient from escaping and creating a risk of exposure to a user of door 110 or an individual in close proximity to door 110. For example, exposure to ultraviolet light can be damaging to the eyes of people or pets and potentially damaging to individuals who have sensitive skin. Furthermore, exposure to ultraviolet light can be harmful to certain objects such as photographic films and some electronics. In a typical embodiment, control box 130 may comprise an indicator such as a light, sign and/or sound that can alert a user of door 110 that germicide source 160 is operating so that people in the area will be able to take additional steps if necessary to avoid exposure to the active ingredient. In an exemplary embodiment, shield 140 may comprise a material that is opaque to ultraviolet light such as a metal sheet or an appropriately selected plastic.

In an exemplary embodiment, the germicide source 160 may be selected to efficiently sterilize the surface of the door handle 150. In an embodiment, an active ingredient may be an ultraviolet light with appropriate characteristics such as the STER-L-RAY™ germicidal lamps that are available from Atlantic Ultraviolet Corporation. In particular, ultraviolet light in the wavelength range of 100-280 nm has been shown to perform well in sterilizing surfaces contaminated with germs and the STER-L-RAY™ lamps emit light at the mercury resonance wavelength of 254 nm.

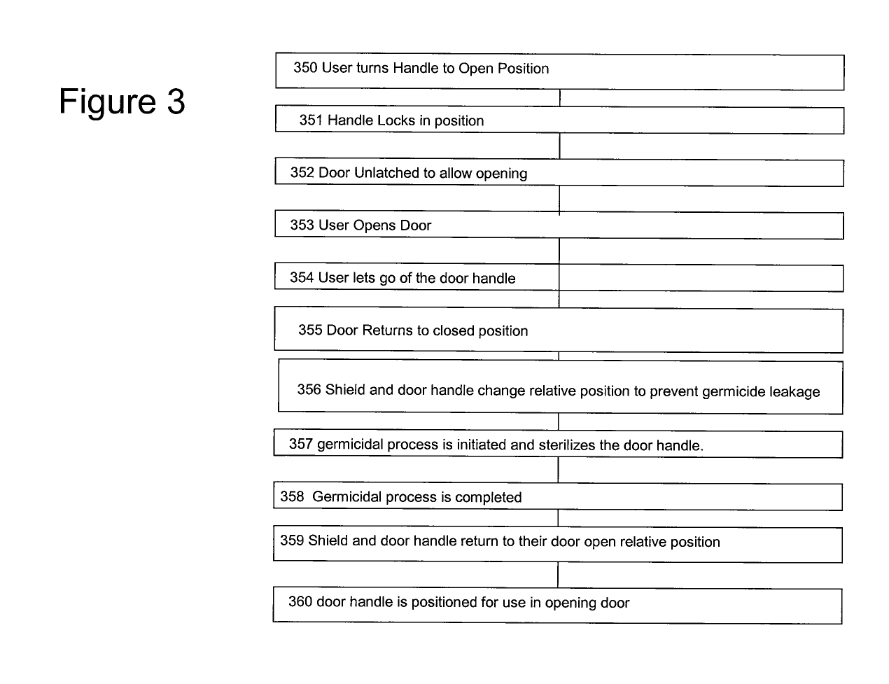

In an exemplary usage scenario a door user will interact with door 110 according to steps identified in FIG. 3. In step 350, a door user turns door handle 150 to an open position. In step 351, door handle 150 locks in the open position. In step 352, door 110 is unlatched to allow opening by the door user. In step 353 the door user opens door 110. In step 354, the door user releases door handle 150. In step 355, door 110 returns to its closed position. In step 356, shield 140 and door handle 150 change their relative position so that door handle 150 is in position for exposure to germicide source 160 while shield 140 prevents leakage.

In step 357, germicide process is initiated to sterilize door handle 150. In step 358, germicide process is complete and sensor system 206 may confirm successful completion of sterilization process. In step 359, shield 140 and door handle 150 return to the door open relative position. In step 360, door handle 150 is made available for use in opening door 110.

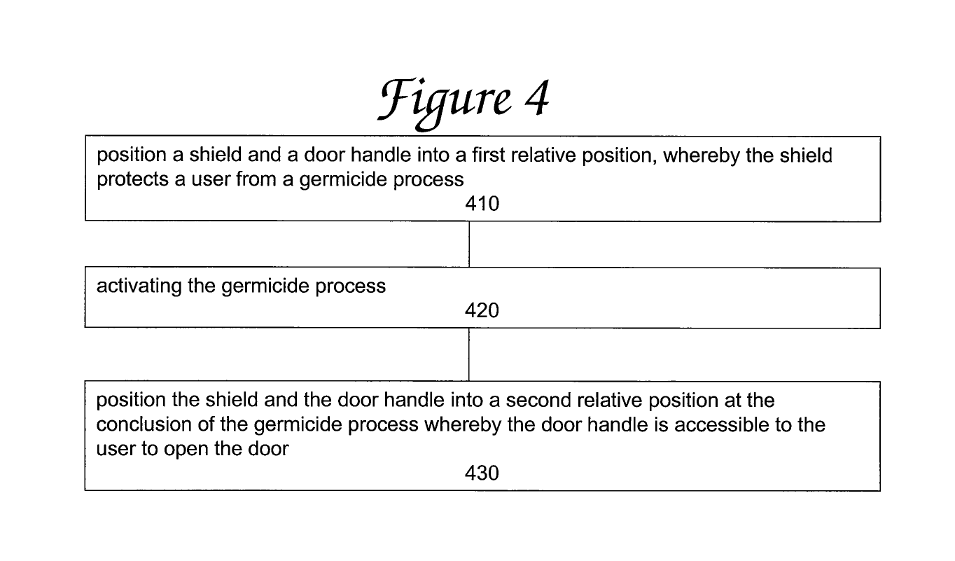

A method as practiced according to an exemplary embodiment is illustrated in FIG. 4. In step 410, a shield and a door handle are moved into a first relative position, whereby the shield protects a user from a germicide process. In step 420, the germicide process is activated. In step 430, the shield and the door handle are moved into a second relative position at the conclusion of the germicide process whereby the door handle is accessible to the user to open the door.

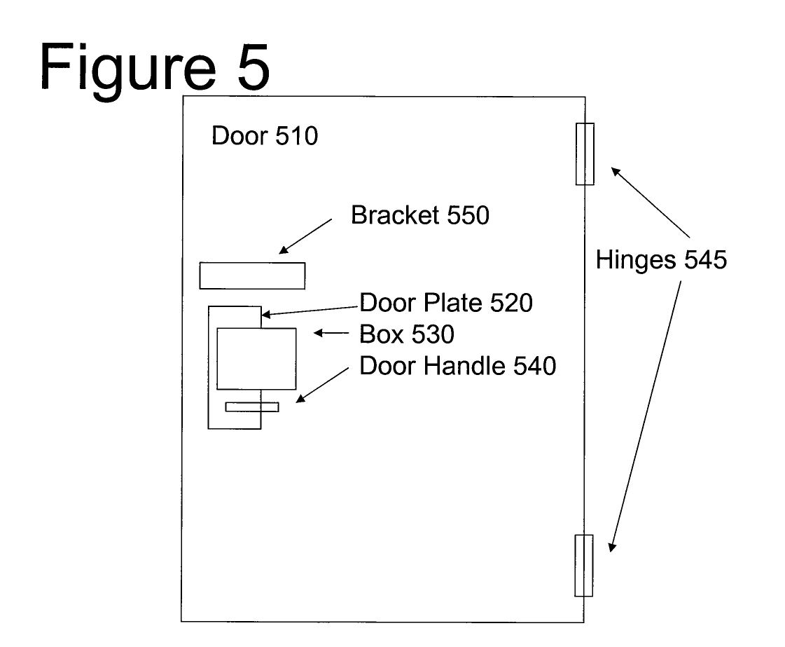

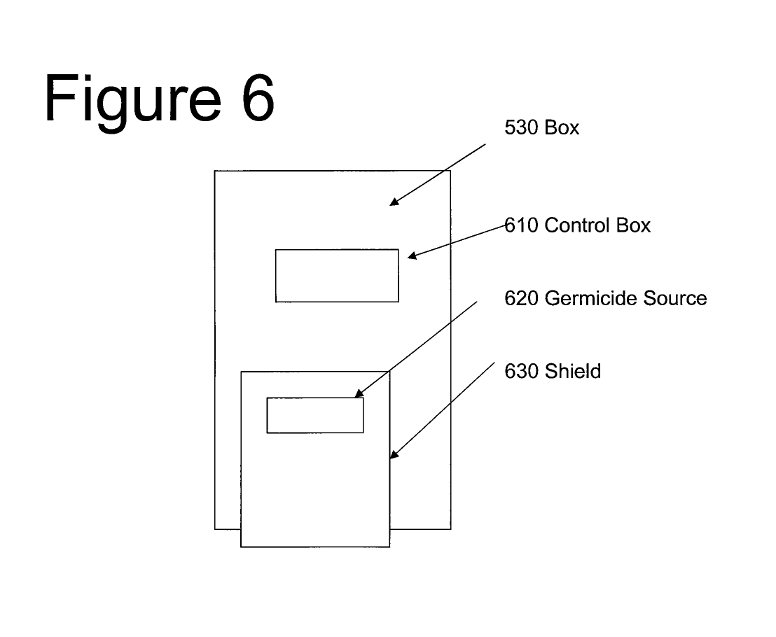

FIG. 5 shows a system comprising a door configured with a door handle sterilizer apparatus mounted via a bracket in accordance with an embodiment. Door 510 which is attached to a structure via hinges 545 comprises door plate 520 and door handle 540. Door 510 is further connected to bracket 550. Bracket 550 is further connected to box 530 which comprises various elements of door handle sterilizer as shown in FIG. 6. Box 530 may be mounted on door 510 via bracket 550. In an embodiment, door handle 540 and door plate 520 may be mounted on an existing door 510 as part of an “after-market” device to provide door handle sterilization to a door that has already been installed in an environment.

FIG. 6 shows a door handle sterilizer apparatus in accordance with an embodiment. Box 530 comprises control box 610, germicidal source 620, and shield 630. Control box 610 is configured to change the relative position of door handle 540 and shield 630 such that a user of door 530 is shielded during application of germicide source 620 during the germicide process. Control box 610 is further operable to retract shield 630 when germicide source 620 is inactive.

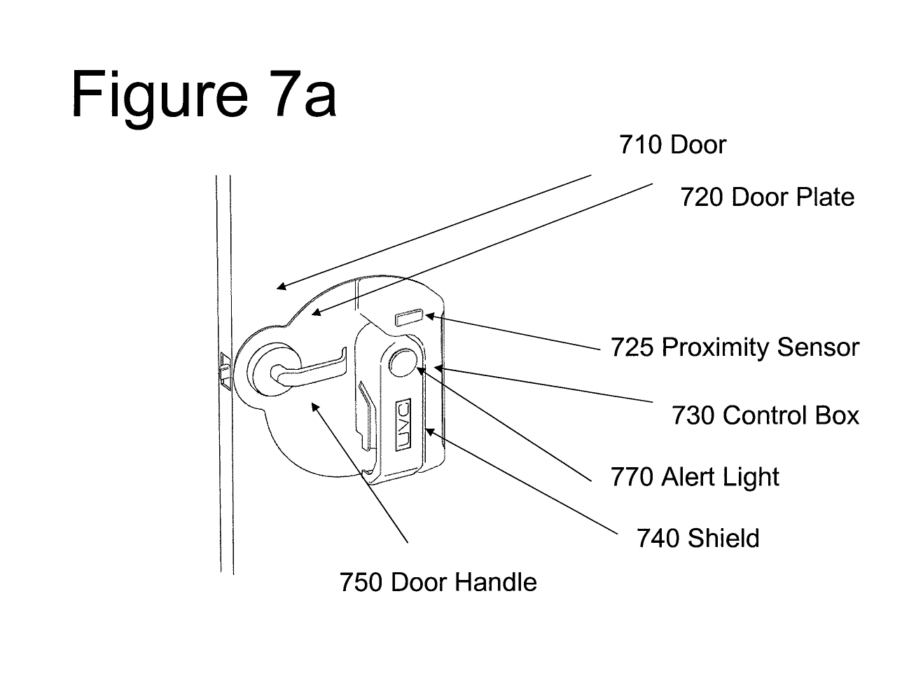

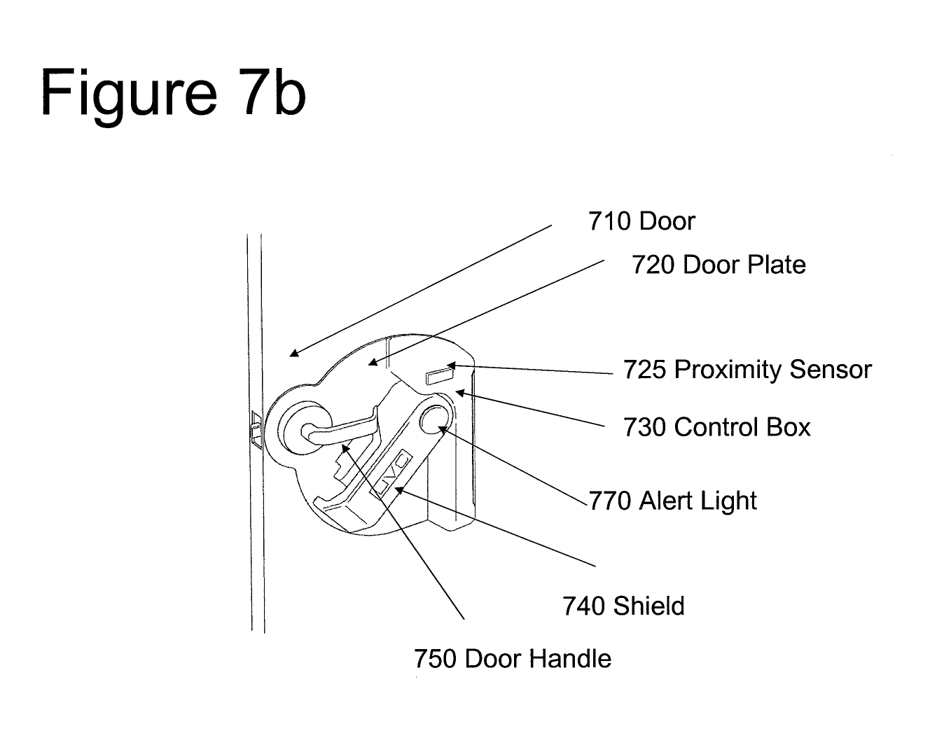

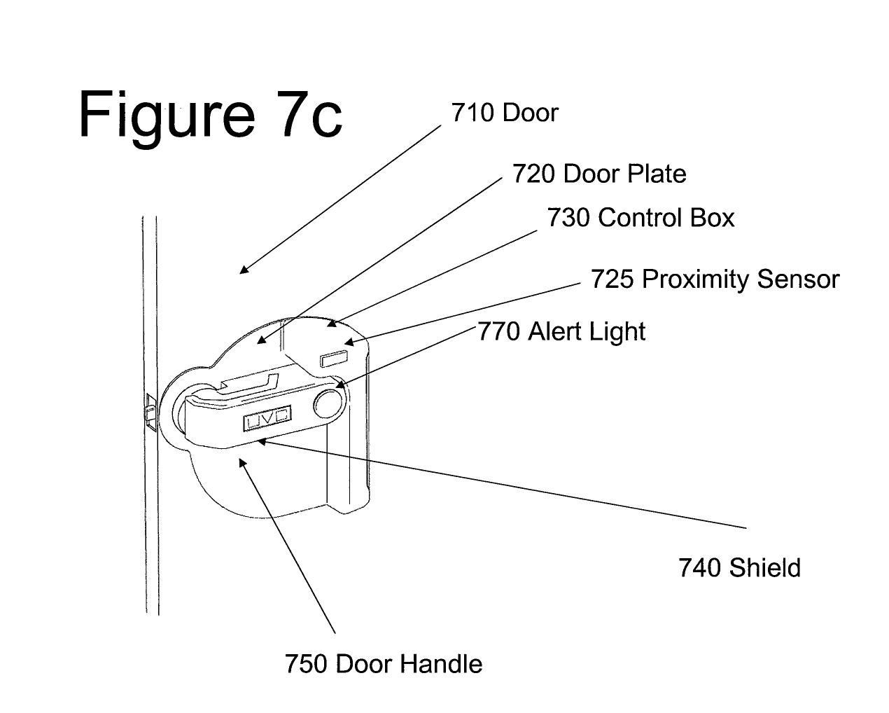

FIGS. 7a-c show several states of a door handle sterilizer apparatus in accordance with an alternative embodiment. FIG. 7a indicates several components of a door handle sterilizer in accordance with this alternative embodiment. Door plate 720 is attached to door 710. Door plate 720 is further attached to control box 730, shield 740, and door handle 750. Proximity sensor 725 is mounted on control box 730. In the alternative embodiment illustrated in FIGS. 7a-c, the germicide source of the door handle sterilizer is fully contained within shield 740. In this alternative embodiment, control box 730 is mechanically connected to one or both of shield 740 and door handle 750.

FIG. 7b illustrates the state of the door handle sterilizer at a moment when shield 740 is rotating in order to change the relative position of shield and door handle. In particular, as shown in FIG. 7b, shield 740 is rotating into a position to cover door handle 750 prior to initiation of the germicide process. FIG. 7c illustrates the door handle sterilizer in accordance with the alternative embodiment where shield 740 has fully rotated into position to cover door handle 750. In accordance with this alternative embodiment, FIG. 7a indicates a moment after door handle 750 has been used by a door user, proximity sensor 725 may detect that no one is in the area of door handle 750. When proximity sensor 725 has detected that no person is in the area of door handle 750, proximity sensor 725 may generate an indication that it is safe to proceed with sterilization of door handle 750. Based on the indication received from proximity sensor 725, control box 730 causes shield 740 to initiate rotation towards a position to cover door handle 750 as shown in FIG. 7b. Once door handle 750 is covered by shield 740 as shown in FIG. 7c, the germicide process is initiated. Upon termination of the germicide process, control box 740 causes shield 740 to rotate in the reverse direction to its original position as shown in FIG. 7a. In this embodiment, alert light 770 may be illuminated to indicate that the door handle is not safe for use. In this embodiment, alert light 770 illuminates when the germicide process has started and turns off at the completion of the germicide process. In certain embodiments, alert light 770 may also be illuminated, for example in a different color, when the door handle has not been sterilized or when the door handle sterilizer is malfunctioning.

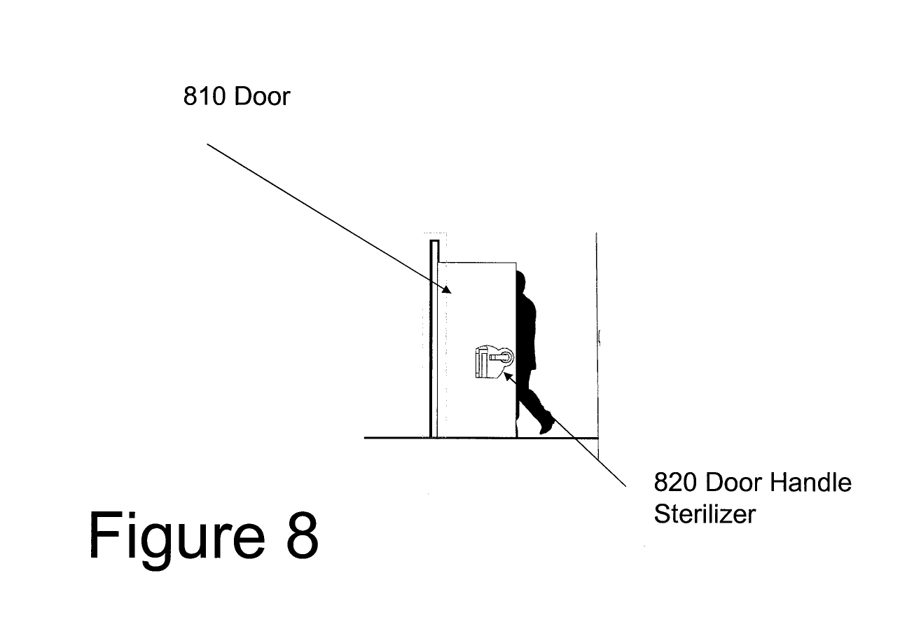

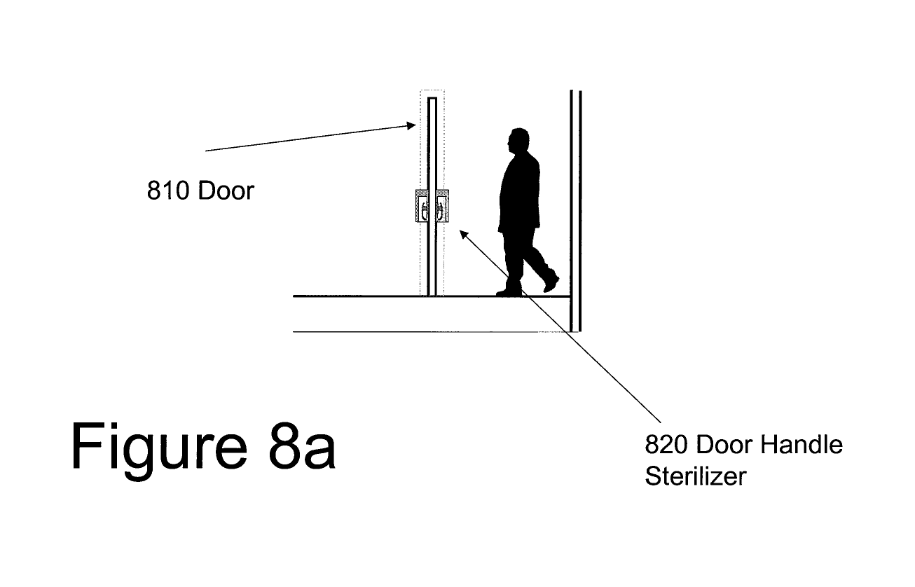

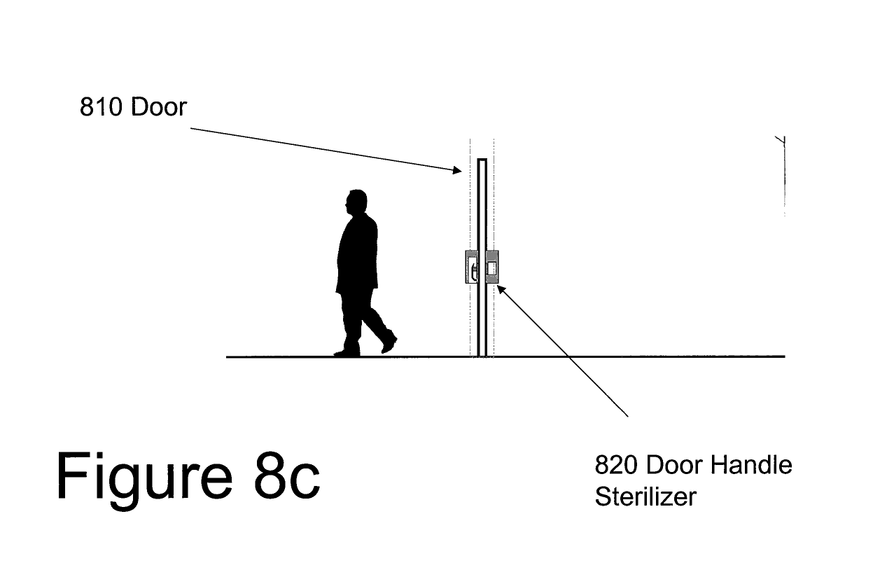

FIGS. 8a-c illustrate a person using a door equipped with a door handle sterilizer in accordance with an embodiment. FIG. 8a shows a person approaching door 810 equipped with door handle sterilizer 820. FIG. 8b shows a person moving through an open door equipped with a door handle sterilizer. At the moment shown in FIG. 8b, the door handle of door handle sterilizer 820 has just been used and is ready to be sterilized. FIG. 8c shows a person having moved through a door equipped with a door handle sterilizer in accordance with an embodiment. FIG. 8c corresponds to a moment when door handle sterilizer 820 may detect that no person is in the area of door handle sterilizer 820. When door handle sterilizer 820 has detected that no person is in the area, door handle sterilizer 820 may commence the germicide process to sterilize the recently used door handle.

In various embodiments, the method steps described herein, including the method steps described in FIG. 4, may be performed in an order different from the particular order described or shown. In other embodiments, other steps may be provided, or steps may be eliminated, from the described methods.

Systems, apparatus, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. For example in an embodiment, control box 130 may act as a client with respect to an external server such as a monitoring server or an email server. Typically, in such a system, the client computers are located remotely from the server computer and interact via a network. The client-server relationship may be defined and controlled by computer programs running on the respective client and server computers.

Systems, apparatus, and methods described herein may be used within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the method steps described herein, including one or more of the steps of FIG. 4. Alternatively, for example, the client or clients may transmit a request adapted to cause a server computer or computers to perform one or more of the method steps described herein, including one or more of the steps of FIG. 4. Certain steps of the methods described herein, including one or more of the steps of FIG. 4, may be performed by a server or by another processor in a network-based cloud-computing system. Certain steps of the methods described herein, including one or more of the steps of FIG. 4, may be performed by a client computer in a network-based cloud computing system. The steps of the methods described herein, including one or more of the steps of FIG. 4, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method steps described herein, including one or more of the steps of FIG. 4, may be implemented using one or more computer programs that are executable by such a processor. A computer program is a set of computer program instructions that may be used, directly or indirectly, in a computer to perform a certain activity or bring about a certain result. A computer program may be written in any form of programming language, including compiled or interpreted languages, and it may be deployed in any form, including as a stand-alone program or as a module, component, subroutine, or other unit suitable for use in a computing environment.

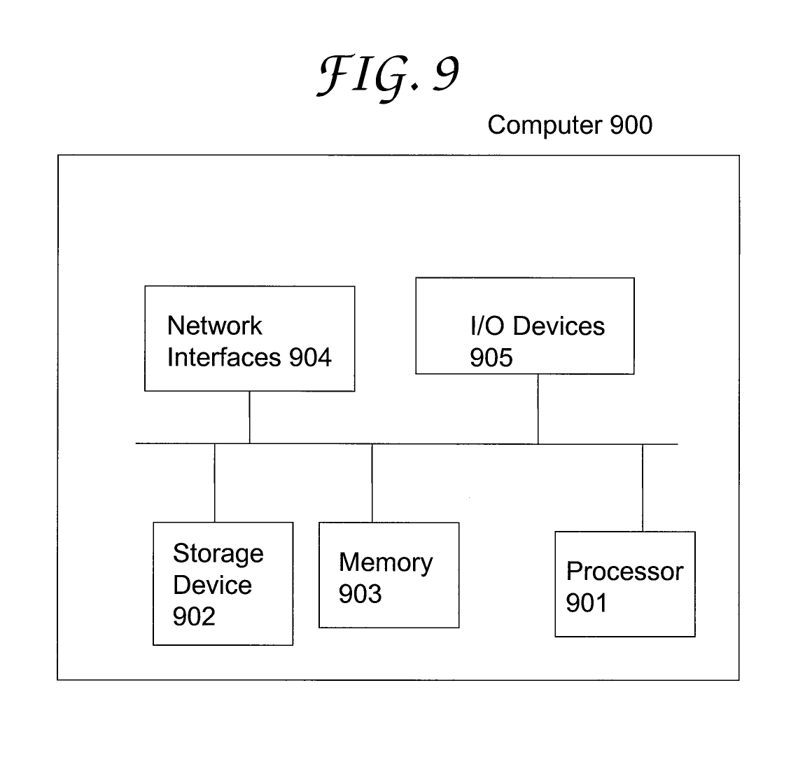

A high-level block diagram of an exemplary computer that may be used to implement systems, apparatus and methods described herein is illustrated in FIG. 9. Computer 900 comprises a processor 901 operatively coupled to a data storage device 902 and a memory 903. Processor 901 controls the overall operation of computer 900 by executing computer program instructions that define such operations. The computer program instructions may be stored in data storage device 902, or other computer readable medium, and loaded into memory 903 when execution of the computer program instructions is desired. Thus, the method steps of FIG. 4 may be defined by the computer program instructions stored in memory 903 and/or data storage device 902 and controlled by the processor 901 executing the computer program instructions. For example, the computer program instructions may be implemented as computer executable code programmed by one skilled in the art to perform an algorithm defined by the method steps of FIG. 4. Accordingly, by executing the computer program instructions, the processor 901 executes an algorithm defined by the method steps of FIG. 4. Computer 900 also includes one or more network interfaces 904 for communicating with other devices via a network. Computer 900 also includes one or more input/output devices 905 that enable user interaction with computer 900 (e.g., display, keyboard, mouse, speakers, buttons, etc.).

Processor 901 may include both general and special purpose microprocessors, and may be the sole processor or one of multiple processors of computer 900. Processor 901 may comprise one or more central processing units (CPUs), for example. Processor 901, data storage device 902, and/or memory 903 may include, be supplemented by, or incorporated in, one or more application-specific integrated circuits (ASICs) and/or one or more field programmable gate arrays (FPGAs).

Data storage device 902 and memory 903 each comprise a tangible non-transitory computer readable storage medium. Data storage device 902, and memory 903, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 905 may include peripherals, such as a printer, scanner, display screen, etc. For example, input/output devices 905 may include a display device such as a cathode ray tube (CRT) or liquid crystal display (LCD) monitor for displaying information to the user, a keyboard, and a pointing device such as a mouse or a trackball by which the user can provide input to computer 900.

In certain embodiments, control box 130, and components thereof, may be implemented using a computer such as computer 900.

One skilled in the art will recognize that an implementation of an actual computer or computer system may have other structures and may contain other components as well, and that FIG. 9 is a high level representation of some of the components of such a computer for illustrative purposes.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the disclosure.

1. A method for sterilizing a door handle comprising:
positioning a shield and a door handle into a first relative position, whereby the shield protects a user from a germicide process;
activating the germicide process; and
positioning the shield and the door handle into a second relative position at the conclusion of the germicide process whereby the door handle is accessible to the user to open the door.

2. The method of claim 1, wherein the germicide process comprises:
illuminating an ultraviolet light.

3. The method of claim 1, wherein positioning a shield and a handle into a first relative position comprises:
moving the shield, while the door handle remains stationary.

4. The method of claim 1, further comprising:
alerting the user that the door handle is accessible to the user to open the door.

5. The method of claim 1, further comprising:
sensing an object in proximity to the door; and
generating a proximity signal based on the sensing.

6. The method of claim 5, further comprising:
activating a safety override system based on the proximity signal.

7. The method of claim 1, further comprising:
receiving information associated with the germicide process; and
transmitting a status message via a network, the status message comprising information associated with the germicide process.

8. A door handle system comprising:
a door handle;
a shield;
a germicide source configured to disinfect the door handle; and
a driver configured to change the relative position of the door handle and the shield such that a user is shielded during application of the germicide source and such that the door handle is accessible to the user when the germicide source is not applied.

9. The system of claim 8, wherein the driver is configured to move the shield from a first position to a second position.

10. The system of claim 8, wherein the germicide source comprises:
an ultraviolet light.

11. The system of claim 8, wherein the driver is configured to move the shield while the door handle remains stationary.

12. The system of claim 8, further comprising:
a sensor configured to detect an object in proximity to the door; and
an interface configured to generate a proximity signal based on the detection of the object.

13. The system of claim 12, further comprising:
a safety override system configured to deactivate the germicide source based on the proximity signal.

14. The system of claim 8, further comprising:
a sensor configured to transmit information associated with the germicide source; and
an interface configured to transmit a status message via a network, the status message comprising information associated with the germicide source.

15. An apparatus configured to attach to a door comprising a door handle, the apparatus comprising:
a bracket configured to attach to the door;
a shield;
a germicide source configured to disinfect the door handle; and
a driver configured to change the relative position of the door handle and the shield such that a user is shielded during application of the germicide source and such that the door handle is accessible to the user when the germicide source is not applied.

16. The apparatus of claim 15, wherein the germicide source comprises:
an ultraviolet light.

17. The apparatus of claim 15, wherein the driver is further configured to move the shield while the door handle remains stationary.

18. The apparatus of claim 15, wherein the driver is further configured to move the shield from a first position to a second position.

19. The apparatus of claim 15, further comprising:
a sensor configured to detect an object in proximity to the door; and
an interface configured to generate a proximity signal based on the detection of the object.

20. The apparatus of claim 19, further comprising:
a safety override system configured to deactivate the germicide source based on the proximity signal.
